(19) United States
(12) Patent Application Publication
(10) Pub. No.: US 2005/0086009 A1
(43) Pub. Date: Apr. 21, 2005

(54) Computer implemented method and system for analyzing genetic association studies

(76) Inventor: David A. Hinds, Mountain View, CA (US)

Correspondence Address: PERLEGEN SCIENCES, INC.;LEGAL DEPARTMENT, 2021 STIERLIN COURT, MOUNTAIN VIEW, CA, 94043, US

(21) Appl. No.: 10/691,069

(22) Filed: Oct. 21, 2003

(51) Int. Cl. G06F 19/00; G01N 33/48; G01N 33/50

(52) U.S. Cl. 702/20

(57) ABSTRACT

A method and system to facilitate the use and application of a sophisticated statistical algorithm to the evaluation and design of case-control genetic association studies. Use of the method and system of the present invention enable the user to relatively easily apply sophisticated statistical analysis to case-control genetic association study design in order to determine whether or not a study will provide a meaningful result before substantial resources are spent.

### BACKGROUND OF THE INVENTION

1. Field of the Invention

The present invention broadly relates to techniques and tools for analyzing case-control genetic association studies for use in biomedical research and clinical drug trials. More specifically, the invention relates to computer implemented methods, media and systems for facilitating the application of a statistical algorithm to the evaluation and/or development of such studies.

2. Description of Related Art

Genetic association studies are conducted to identify a correlation or “association” between a region or regions of a genome represented by a gene or genes and a disease or other phenotypic trait. Regions of a genome may include genes, regulatory regions, etc. Genetic association studies are of various types including cohort studies, family studies and case-control studies.

Cohort studies are prospective. A large population group is randomly selected for study. Individuals is the population who have or acquire a phenotypic trait of interest (e.g., a disease) are designated “incident cases.” However, this type of study has a number of drawbacks that make it unappealing in practice. It is very difficult to establish a dependable control population due to the variable age of onset and multi-variable causation of many traits of interest. Also, very large populations are typically required in order to include sufficient incident cases to obtain a statistically significant result when the study data is analyzed. Thus, these studies, while potentially a source of very valuable information, are extremely costly and, as a result, often disfavored by drug researchers.

Family studies are retrospective and use classic genetic epidemiology techniques to analyze collections of genetic data from families having members with a particular phenotypic trait of interest. Advantages of this type of study are that it is generally possible to reduce the variability of other factors in the control population, and the applicable Mendelian analytical techniques are well known and understood. This type of study is also limited, however, by the willingness of families to participate and the multi-factorial nature of many traits of interest.

Case-control studies are also retrospective studies, but rather than using family populations or randomly selected populations, such a study uses a population of unrelated (or not necessarily related) people with a particular trait (“cases”). These cases are compared with a group of people who do not have the trait (“controls”). The studies generally involve establishing values for a number of trait parameters which are then subjected to statistical manipulation to determine whether or not there is a different distribution of common haplotypes between the case and control populations for the gene(s) (locus or loci) of interest. The results of these studies can establish a link between a gene and a disease and this information can then be used in a process to discover drugs to treat or prevent the disease.

One difficulty encountered with genetic association studies is that if the study parameters are not correctly tailored, it may not be possible to obtain a statistically significant result. It is desirable to ensure that a study design will provide statistically significant results before the study is undertaken in order to avoid wasting the significant time, effort and financial resources required to conduct such studies.

Statistical techniques are available to evaluate a genetic association study design for the statistical significance of its results. However, the complex mathematics involved in the application of such techniques render them practically unusable for many of the biological scientists, generally geneticists and epideimiologists, designing and conducting the studies. As a result, this type of analysis of a study design is often not undertaken, resulting in resources being spent on studies that have little or no chance of giving a useful (statistically valid) result.

Computer programs have been developed in order to facilitate the application of statistical analysis to genetic association study design. For example, the Statistical Analysis for Genetic Epidemiology (S.A.G.E.) software package available from Case Western Reserve University provides tools for facilitating the application of statistical analysis in the context of family (sib-based) studies. However, to date, tools available to facilitate the application of the more complex statistical analysis involved in case-control genetic association studies are lacking.

Accordingly, what is needed is a way to facilitate analysis of genetic association case-control study designs in order to optimize the use of resources available for conducting such studies by ensuring that the results obtained from the studies undertaken will be statistically valid.

### SUMMARY OF THE INVENTION

This invention provides a method and system to facilitate the use and application of a sophisticated statistical algorithm to the evaluation and design of genetic association case-control studies. Use of the method and system of the present invention enable the user to relatively easily apply sophisticated statistical analysis to genetic association case-control study design in order to determine whether or not a study will provide a meaningful result before substantial resources are spent.

In one aspect, the invention pertains to a method for analyzing a genetic association case-control study. The method involves providing a spreadsheet program running on a computer, and programming the spreadsheet software with a statistical “power” algorithm configured to analyze a genetic association case-control study. Values for parameters defining the genetic association case-control study are input to the spreadsheet program, and the study's power to detect a significant difference in distribution of observed allele frequency in cases and controls for the input parameter values is then determined using the power algorithm. The power is also generally displayed in the spreadsheet.

A plurality of values may be input for one or more parameters and the determined power displayed in the spreadsheet for each. The power results obtained may be displayed in graphical form to facilitate interpretation and use of the results.

In an alternative embodiment, the present invention may also be used to determine selected parameter values defining a genetic association case-control study from a desired or required power for the study. According to this aspect of the invention, a computer implemented method for analyzing a genetic association case-control study is provided. The method involves providing a spreadsheet program running on a computer, and programming the spreadsheet software with a statistical power algorithm configured to analyze a genetic association case-control study. A subset of values for parameters defining the genetic association case-control study and a desired power of the study to detect a significant difference in distribution of observed allele frequency in cases and controls are input to the spreadsheet program. Then, a complete set of values for parameters defining the genetic association case-control study are determined using the power algorithm.

In other aspects, the invention pertains to systems for implementing the method of the invention and computer-readable media bearing instructions for conducting the method of the invention.

These and other features and advantages of the present invention are described below where reference to the drawings is made.

### BRIEF DESCRIPTION OF THE DRAWINGS

FIG. 1A provides a basic illustration of a computer implemented spreadsheet suitable for implementation of the present invention.

FIG. 1B provides a partial illustration of a computer implemented spreadsheet programmed for implementation of the present invention.

FIGS. 2A and B illustrate a computer system suitable for implementing embodiments of the present invention.

FIG. 3 illustrates a process flow for a computer implemented method for analyzing a genetic association case-control study in accordance with the present invention.

FIG. 4A illustrates a column showing a baseline set a parameter values for a genetic association case-control study involving a hypothetical gene.

FIG. 4B shows the baseline values of FIG. 4A and several additional columns containing entries for a range of other values for the QTL frequency and Marker frequency parameters so that the effect of changing these parameters can been seen input into a computer-based spreadsheet programmed with a power algorithm in accordance with the present invention.

FIG. 4C is a plot of power vs. allele frequency illustrating the change of power depending upon QTL and marker frequencies for the hypothetical gene/marker pair for the data in the spreadsheet illustrated in FIG. 4B.

FIG. 5A illustrates sets of parameter values for a genetic association study involving a hypothetical gene input into a computer-based spreadsheet programmed with a power algorithm in accordance with the method and system of the present invention.

FIG. 5B is a plot of power vs. heritability data from the spreadsheet of FIG. 5A illustrating the change of power depending upon broad-sense heritability for a hypothetical gene.

FIG. 6A illustrates sets of parameter values for a genetic association study involving a hypothetical gene input into a computer-based spreadsheet programmed with a power algorithm in accordance with the method and system of the present invention.

FIG. 6B is a plot of power vs. dominance effect data from the spreadsheet of FIG. 6A illustrating the change of power depending upon dominance effect for a hypothetical gene.

FIG. 7 is a plot of power vs. number of cases for data obtained from a spreadsheet programmed with a power algorithm in accordance with the method and system of the present invention, illustrating the change of power depending upon a measure of association between a hypothetical gene and its marker (Lewontin's D′).

### DETAILED DESCRIPTION OF SPECIFIC EMBODIMENTS

Reference will now be made in detail to specific embodiments of the invention. Examples of the specific embodiments are illustrated in the accompanying drawings. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail in order not to unnecessarily obscure the present invention.

### I. INTRODUCTION

As noted above, statistical techniques are available to evaluate a genetic association study design for the statistical significance of its results. Statistical “power” is a measure of a study's ability to detect what is desired to be detected given the specific parameters of the study. In the context of a genetic association case-control study, what is generally desired to be detected is a significant difference in distribution of observed allele (or, haplotype) frequency in cases and controls for a gene of interest. The application of power algorithms to genetic association study design is discussed in Risch, N. and Teng, J. (1998) The Relative Power of Family-Based and Case-Control Designs for Linkage Disequilibrium Studies of Complex Human Diseases: 1. DNA Pooling, Genome Research 8:1273-1288, the disclosure of which is incorporated by reference herein in its entirety and for all purposes. However, these statistical power calculations are sufficiently mathematically complex that they are practically unusable for many of the biological scientists designing and conducting the studies. As a result, this type of analysis of a study design is often not undertaken, resulting in resources being spent on studies that have little or no chance of giving a statistically valid result. The present invention provides a method and system to facilitate the use and application of a complex statistical power analysis to the evaluation and design of genetic association case-control studies.

The present invention uses a computer-based spreadsheet program and associated method to facilitate the use and application of a statistical power algorithm to the analysis of genetic association case-control studies. Schork et al. describe a technique of linkage disequilibrium (LD) mapping applied to the analysis of human quantitative trait loci and investigate the power of this method for some hypothetical gene-effect scenarios (Schork, N. J., Nath, S. K., Fallin, D. and Chakravarti, A. (2000) Linkage Disequilibrium Analysis of Biallelic DNA Markers, Human Quantitative Trait Loci, and Threshold-Defined Case and Control Subjects, Am. J Hum. Genet. 67:1209-1218, incorporated by reference herein in it entirety). The spreadsheet-based tool and method of the present provides a user-friendly format for a study designer or evaluator to input study parameters, apply the statistical calculations required to analyze the statistical validity of the study, and receive and review the results. The statistical power calculations of Schork et al., for example, are adapted to an algorithm for implementation in the spreadsheet facilitating its use by genetic association case-control study designers. Use of the method and system of the present invention enable the user to relatively easily apply the sophisticated statistical analysis to genetic association case-control study design in order to determine whether or not the study will provide a meaningful result before substantial resources are spent.

A description of a statistical power algorithm suitable for genetic association case-control study analysis and adapted for use in a computer implemented method and system in accordance with the present invention follows. In addition, software and hardware for implementation of the power algorithm for genetic association study analysis are described. Also, further details and examples of genetic association case-control study analysis in accordance with the present invention are provided.

### II. POWER ALGORITHM

As noted above, statistical “power” is a measure of a study's ability to detect what is desired to be detected given the specific parameters of the study. Power is related to the conclusion of a statistical test. In a case-control study there are four possible outcomes of a statistical test: 1) it is concluded there is a difference between a case and control when in fact there is; 2) it is concluded there is a difference when in fact there is none (this is referred to as false positive or type 1 error, α); 3) it is concluded there is no difference between a case and control when in fact there is none; and 4) it is concluded there is no difference when in fact there is (this is referred to as false negative or type II error, β). There is an inverse relationship between achievable type I (α) and type II (β) error rates for a given experimental design. Power may be expressed as: Power=1−β. Therefore, the higher the value of power, the lower the rate of false negatives and the more detectable the difference desired to be detected.

A genetic association study involves comparisons of frequencies of an allele or haplotype between individuals with and without a phenotypic trait of interest. If compelling evidence for frequency differences exist, then the locus or loci in question harbors alleles that directly (causally) or indirectly (via alleles at a neighboring locus or loci) influence the trait in question. Recently, experimental methods have become available that enable large scale association studies of complex, multifactorial traits, with acceptable type I and type II error rates. The parameters defining these studies may be the number of cases, the number of controls per case, trait value thresholds used to define the cases and controls, and a desired type 1 (false positive) error rate. The results of these studies may establish a link between a gene and a disease and this information can then be used in a process to discover drugs to treat or prevent the disease.

The invention involves adaptation of these statistical power calculations to an algorithm for implementation in a spreadsheet program running on a computer. The algorithm identifies formulas for calculating various features based on the input data values for the parameters defining the genetic association case-control study. In a preferred embodiment, the statistical power calculations are applied to a genetic association study as described below.

A quantitative trait is modeled as in Schork et al., such that for a quantitative trait locus (QTL), the ‘bb’ genotype has a mean trait value of −a, the ‘BB’ genotype has a mean trait value of +a, and the heterozygote ‘Bb’ has a mean trait value of d. For individuals with the same genotype, the trait is defined to have a standard deviation of 1. The complete population is a mixture of people drawn from the trait distributions for these three genotypes.

Given:
QTL frequency of ‘B’allele, p
Broad-sense heritability attributed to this locus, H
Relative dominance effect for this locus, d/a
lower tail area of the trait distribution represented by controls, a1
upper tail area of the trait distribution represented by cases, a2

First, to determine the additive effect, a, the following definitions are used:
Additive variance, Va=2p(1−p) (a−(2d−1))\hat{ }2
Dominance variance, Vd=(2p(1−p)d)\hat{ }2
Total genetic variance, Vg=Va+Vd
Broad-sense heritability, H=Vg/(Vg+1)
   This gives an expression for H in terms of p, a, and d. This can be solved for a, in terms of H,p, and d/a:
   a=sqrt[(H/(1−H))/(2p(1−p)[(1−(d/a)(2p−1))\hat{ }2+2p(1−p)(d/a)\hat{ }2])]

Trait value cutoffs for the case and control populations based on the population distribution of the trait are then determined. That distribution can be determined from the known means for each of the three possible genotypes, and the known frequency of each genotype in the population:

| | mean | frequency |
|---|---|---|
| 'bb' | -a | (1 − p)\hat{ }2 |
| 'Bb' | d | p(1 − p) |
| 'BB' | +a | p\hat{ }2 |

Given a trait threshold t, the probabilities of having a trait value less than t and a given genotype ‘bb’, ‘Bb’ or ‘BB’ can be determined:
p(x<t, bb)=Φ(t+a) (1−p)\hat{ }2
p(x<t, Bb)=Φ(t−d) p(1−p)
p(x<t, BB)=Φ(t−a) p\hat{ }2
where Φ(x) is the cumulative normal distribution function, the area under a normal distribution integrated from negative infinity to x. From this, the total probability of having a trait value less than t is:
p(x<t)=p(x<t, bb)+p(x<t, Bb)+p(x<t, BB)

The trait value cutoff for controls is the value for which p(x<t)=a1, and the cutoff for cases is the value for which p(x>t)=a2. This equation is solved numerically by iteratively varying t until the resulting value of p(x<t) matches the target value. Alternatively, a binary search strategy may be used to solve the same problem. In one embodiment, the iterative solution is determined using the “goal seek” function in Microsoft Excel™. From this numerical solution of the p(x<t) equation, trait cutoffs t1 and t2 are obtained from a1 and a2, such that p(x<t1)=a1, and p(x>t2)=a2.

Given t1 and t2, the frequencies of the B allele among the control and case populations may be calculated as follows:
p(B|x<t1)=[0.5*p(x<t1, Bb)+p(x<t1, BB)]/p(x<t1)
p(B|x>t2)=[p\hat{ }2+p(1−p)−0.5*p(x<t2, Bb)−p(x<t2, BB)]/p(x>t2)
The first equation is relatively straightforward. The second one is more complicated because p(x<t2, Bb) has been explicitly evaluated rather than p(x>t2, Bb) which is what is really required.

An additional complication is that the site being genotyped may not be the same as the functional site; it may only be correlated with the functional site. Say the marker that is genotyped has alleles ‘M’ and ‘m’, with ‘M’ associated with the high-risk ‘B’ allele. A common measure of association between polymorphic sites is “Lewontin's D′” which is a scaled correlation so that a value of 0 means no association and 1 means the maximum possible association for a pair of markers, given their (possibly different) allele frequencies:

Given:
Allele frequency of ‘M’ allele, s
Disequilibrium between marker and QTL, D′
   the marker allele frequencies for controls and cases may be expressed as:
   p1=p(M|x<t1)=s+D′[p(B|x<t1)−p]min((1−s)/(1−p), s/p)
   p2=p(M|x>t2)=s+D′[p(B|x>t2)−p]min((1−s)/(1−p), s/p)

Having the expected allele frequencies in the controls (p1) and cases (p2), the “power” to detect this difference can be determined by determining how likely it is that a sufficiently high score would be obtained for the observed difference given a desired false positive (i.e., Type I error) rate and the numbers of cases and controls to be in the pools:

Given:
Number of cases, N
Number of controls per case, c
Desired type I error rate, α
   The study's power is determined as:
   p′=(c*p1+p2)/(1+c)
   Φ(Zα)=1−α
   power=1−β=Φ[sqrt[2n(p1−p2)\hat{ }2/((1+1/c) p′(1−p′))]−Zα]
   The factor of 2 arises in the final equation because the effective sample size for n individuals is 2n alleles, since a person has two copies of each (non-sex) chromosome.

From the trait distributions, the penetrance, i.e., the probability of being affected given a known genotype, of the “case” phenotype for each possible QTL genotype can also be determined.
f0=p(x>t2|bb)=Φ(t2+a)
f1=p(x>t2|Bb)=Φ(t2−d)
f2=p(x>t2|BB)=Φ(t2−a)

Two common measures of the magnitude of effect of a genetic locus are its population attributable risk, and its genotype relative risk. The population attributable risk is the fraction of all cases who would not be cases if their genotypes were all converted to the lowest-risk genotype at this locus (i.e., if all ‘bB’ and ‘BB’ cases were converted to ‘bb’). It is a measure of the therapeutic impact of eliminating the high-risk allele. The genotype relative risk is the increased odds of being a case for the ‘bB’ genotype compared to the ‘bb’ genotype. In terms of the penetrance values, the population attributable risk and genotype relative risk are given by:
PAR=1−f0/[f2*p\hat{ }2+f1*p(1−p)+f0*(1−p)\hat{ }2]
GRR=f1/f0

Thus adapted to a case-control genetic association study, the statistical power algorithm is implemented in spreadsheet software running on a computer system to facilitate its use.

### III. IMPLEMENTATION

The present invention may be implemented, in whole or in part, on a computing apparatus running spreadsheet software. Spreadsheet software and its operation is well known and will not be described in detail in order not to obscure the present invention. Well known examples of spreadsheet software include Lotus 1-2-3™ available from International Business Machines Corporation and Excel™ available from Microsoft Corporation. Briefly, spreadsheet software simulates a paper spreadsheet or worksheet which appears on the screen as a matrix of rows and columns, the intersections of which are referred to as cells. The user can scroll horizontally or vertically across the spreadsheet to view the cells. The cells contain labels, numeric values or mathematical formulas which command the spreadsheets program to perform calculations. The formulas entered in the cells perform the calculations using the entered labels (variables) and numeric values and possibly the results obtained from other formulas entered in the spreadsheet. In this way, complex mathematical operations can be rendered more practically usable.

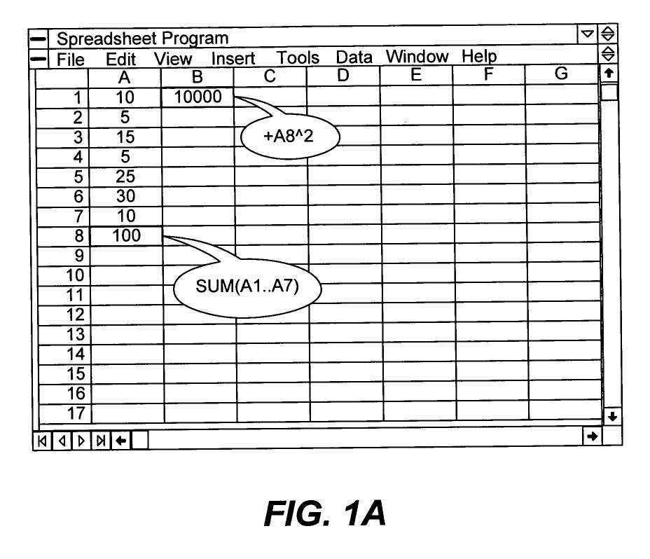

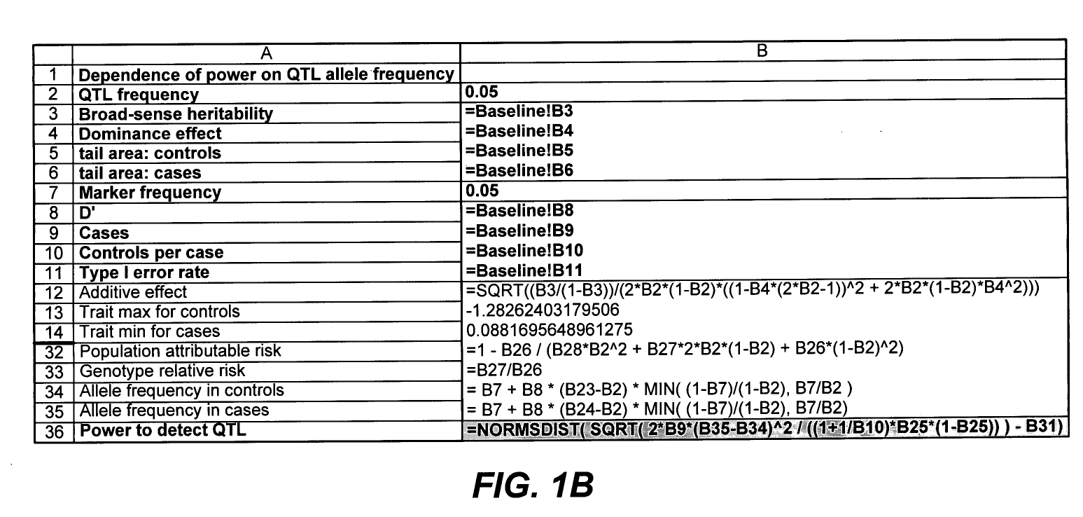

FIG. 1A provides a basic illustration of a computer implemented spreadsheet. Cells labeled A1 through A7 contain numeric values. Cell A8 contains a formula ordering a summing operation on the values in cells A1 through A7. The sum is displayed in the cell. Cell B1 contains a formula ordering a squaring operation on the sum in cell A8. Again, the result is displayed in the cell. Formulas having far more complexity may be entered into cells in a spreadsheet to accomplish complicated calculations, such as are required in genetic association study analyses.

For implementation of the present invention, a user will enter the labels, values and formulas for the statistical power algorithm described above into cells of a spreadsheet program running on a computer. Many of the values, in particular those for the genetic factors (trait value thresholds) used to define case and controls will be assumptions based on the best available information and scientific judgment of the user. These trait value thresholds used to define case and controls include a quantitative trait locus (QTL) frequency; broad sense heritability; dominance effect; case and control tail areas; marker frequencies; and a measure of association between marker and QTL sites (e.g., Lewontin's D′). The number of cases, a ratio of controls to cases, and a desired false positive (Type I) error rate are also input. The power of the genetic association study defined by the input data is then determined by the power algorithm and the results output in the appropriate cell (programmed with the formula for power from the power algorithm) of the spreadsheet.

Various other features of the input data, generally intermediates in the power determination, may also be determined and output in appropriate cells programmed with the respective formulas for these features, in accordance with the power algorithm. These include additive effect; maximum and minimum trait values for controls and cases, respectively; population attributable risk, genotype relative risk, and allele frequency in cases and controls.

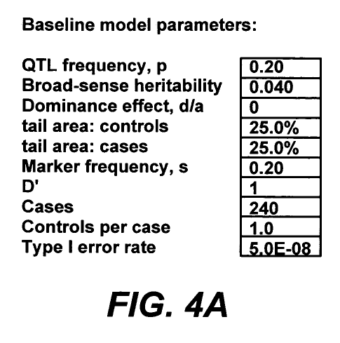

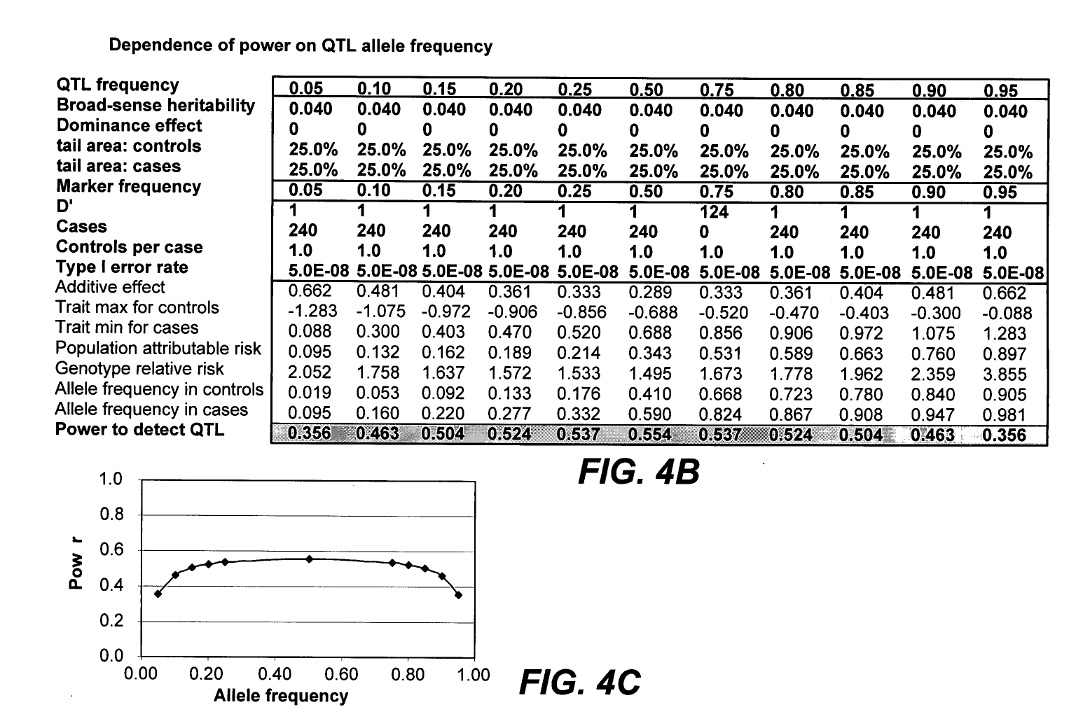

An example of the programming of a spreadsheet in accordance with the present invention is illustrated in FIG. 1B. The figure shows the formulas entered in the cells of a representative column of a spreadsheet in accordance with the present invention with the alpha-numeric notations referencing the data in other cells in the spreadsheet ((e.g, B3 references the data in column B, row 3). Values for the parameters defining the case-control study (quantitative trait locus (QTL) frequency; broad sense heritability; dominance effect; case and control tail areas; marker frequencies; Lewontin's D′; the number of cases, a ratio of controls to cases, and a desired false positive (Type I) error rate) are entered from a baseline table, such as depicted in FIG. 4A of the Example, below. The formulas for features of the input parameter data, are then entered (programmed) in appropriate cells, in accordance with the power algorithm. The formulas for the features additive effect; population attributable risk, genotype relative risk, allele frequency in cases and controls, and power), described and elucidated above in the power algorithm, are shown in their respective cells in the representative column of FIG. 1B. Actual values are shown in the spreadsheet depicted in FIG. 4B in the Example, below.

Many spreadsheets integrate charting, plotting and database functionalities which are useful in some embodiments of the present invention, particularly for displaying the results of the statistical calculations. The tabulated results of the case-control genetic association study analyses of the present invention may preferably be displayed using other spreadsheet functionalities, in particular in graphical form. Also, many spreadsheets have a graphical interface with pull down menus and a point and click capability using a mouse pointing device to facilitate navigation and data input and retrieval. A preferred spreadsheet for implementation of the preset invention is the Excel™ spreadsheet software program available from Microsoft Corporation. Further details on the capabilities and operation of Excel™ are available from a variety of Microsoft and third party publications, for example, Frye, Curtis. Microsoft Excel Version 2002 Step by Step, Microsoft Press (2001), incorporated by reference herein.

Useful machines for supporting the spreadsheet software and performing the operations of this invention include general purpose digital computers or other data processing devices. Such apparatus may be specially constructed for the required purposes, or it may be a general purpose computer selectively activated or reconfigured by a computer program stored in the computer. The processes presented herein are not inherently related to any particular computer or other apparatus. In particular, various general purpose machines may be used with programs written in accordance with the teachings herein, or it may be more convenient to construct a more specialized apparatus to perform the required method steps. The required structure for a variety of these machines will appear from the description given above.

Certain aspects of the methods of the present invention may be embodied in computer software code. Accordingly, the present invention relates to machine readable media that include program instructions, data, etc. for performing various operations described herein. Examples of machine-readable media include, but are not limited to, magnetic media such as hard disks, floppy disks, and magnetic tape; optical media such as CD-ROM disks; magneto-optical media such as floptical disks; and hardware devices that are specially configured to store and perform program instructions, such as read-only memory devices (ROM) and random access memory (RAM). The invention may also be embodied in a carrier wave traveling over an appropriate medium such as airwaves, optical lines, electric lines, etc. Examples of program instructions include both machine code, such as produced by a compiler, and files containing higher level code that may be executed by the computer using an interpreter.

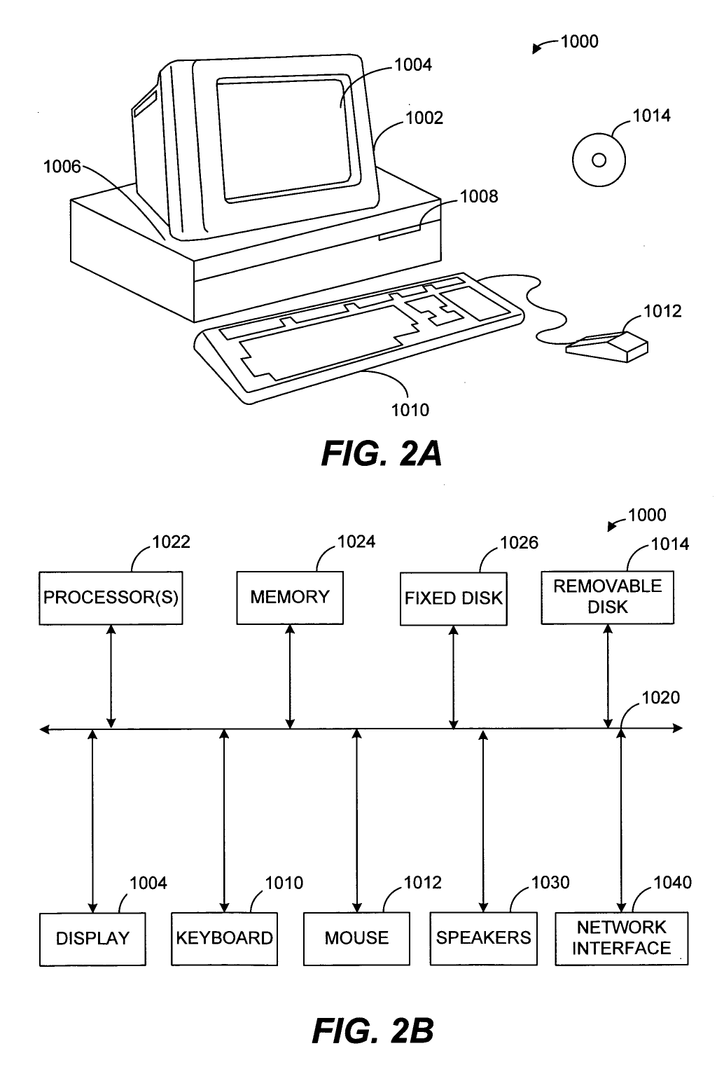

FIGS. 2A and B illustrate a computer system 1000 suitable for implementing embodiments of the present invention. FIG. 2A shows one possible physical form of the computer system. Of course, the computer system may have many physical forms ranging from an integrated circuit, a printed circuit board and a small handheld device up to a huge super computer. Computer system 1000 includes a monitor 1002, a display 1004, a housing 1006, a disk drive 1008, a keyboard 1010 and a mouse 1012. Disk 1014 is a computer-readable medium used to transfer data to and from computer system 1000.

FIG. 2B is an example of a block diagram for computer system 1000. Attached to system bus 1020 are a wide variety of subsystems. Processor(s) 1022 (also referred to as central processing units, or CPUs) are coupled to storage devices including memory 1024. Memory 1024 includes random access memory (RAM) and read-only memory (ROM). As is well known in the art, ROM acts to transfer data and instructions uni-directionally to the CPU and RAM is used typically to transfer data and instructions in a bidirectional manner. Both of these types of memories may include any suitable of the computer-readable media described below. A fixed disk 1026 is also coupled bi-directionally to CPU 1022; it provides additional data storage capacity and may also include any of the computer-readable media described below. Fixed disk 1026 may be used to store programs, data and the like and is typically a secondary storage medium (such as a hard disk) that is slower than primary storage. It will be appreciated that the information retained within fixed disk 1026, may, in appropriate cases, be incorporated in standard fashion as virtual memory in memory 1024. Removable disk 1014 may take the form of any of the computer-readable media described below.

CPU 1022 is also coupled to a variety of input/output devices such as display 1004, keyboard 1010, mouse 1012 and speakers 1030. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, or other computers. CPU 1022 optionally may be coupled to another computer or telecommunications network using network interface 1040. With such a network interface, it is contemplated that the CPU might receive information from the network, or might output information to the network in the course of performing the above-described method steps. Furthermore, method embodiments of the present invention may execute solely upon CPU 1022 or may execute over a network such as the Internet in conjunction with a remote CPU that shares a portion of the processing. The above-described devices and materials will be familiar to those of skill in the computer hardware and software arts.

Because program instructions may be employed to implement the methods and systems described herein, the present invention relates to machine readable media that include program instructions, data, etc. for performing various operations described herein. Examples of machine-readable media include, but are not limited to, magnetic media such as hard disks, floppy disks, and magnetic tape; optical media such as CD-ROM disks; magneto-optical media such as floptical disks; and hardware devices that are specially configured to store and perform program instructions, such as read-only memory devices (ROM) and random access memory (RAM). The invention may also be embodied in a carrier wave travelling over an appropriate medium such as airwaves, optical lines, electric lines, etc. Examples of program instructions include both machine code, such as produced by a compiler, and files containing higher level code that may be executed by the computer using an interpreter.

### IV. GENETIC ASSOCIATION STUDY ANALYSIS

In accordance with the present invention, a user will enter the labels, values and formulas for the statistical power algorithm described above into cells of a spreadsheet program running on a computer. Many of the values, in particular those for the genetic factors (trait value thresholds) used to define case and controls will be assumptions based on the best available information and scientific judgment of the user. These trait value thresholds used to define case and controls include a quantitative trait locus (QTL) frequency; broad sense heritability; dominance effect; case and control tail areas; marker frequencies; and a measure of association between marker and QTL sites (e.g., Lewontin's D′). The number of cases, a ratio of controls to cases, and a desired false positive (Type I) error rate are also input. The power of the genetic association study defined by the input data is then determined by the power algorithm and the results output in the spreadsheet. The results may also preferably be displayed using other spreadsheet functionalities, in particular in graphical form.

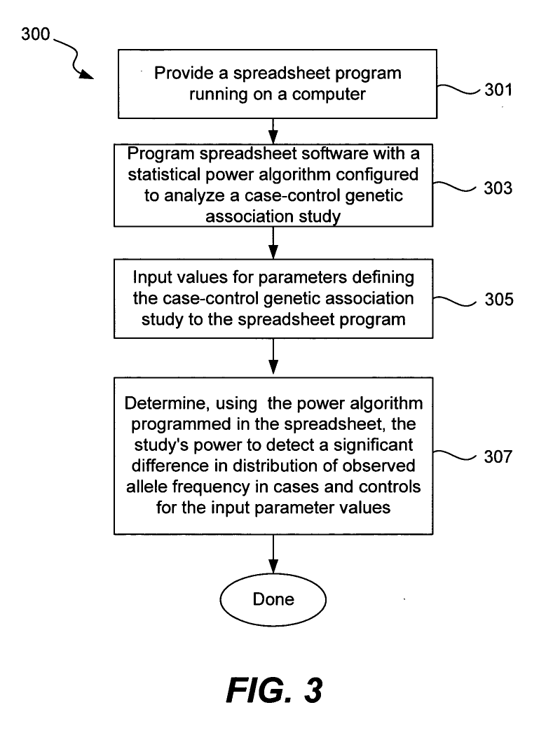

FIG. 3 illustrates a process flow for a computer implemented method for analyzing a case-control genetic association study. The method is implemented by a power algorithm, such as described above, programmed into spreadsheet software running on a computer.

The method 300 begins with the provision of a spreadsheet program running on a computer, such as described above (301). The spreadsheet software is programmed with a statistical power algorithm, configured to analyze a case-control genetic association study, such as described above (303). In this regard, formulas for calculating various features based on the data values for the parameters defining the genetic association case-control study are input into cells of the spreadsheet. The values for parameters defining the genetic association case-control study are also input to the spreadsheet program by a user (305). Of course, steps 303 and 305 can be performed in any order. The spreadsheet performs the power algorithm to determine the study's power to detect a significant difference in distribution of observed allele frequency in cases and controls for the input parameter values and outputs the result(s) in the appropriate cell(s) (programmed with the formula for power from the power algorithm) (307).

The spreadsheet typically has a tabular interface displayed on the computer monitor screen to facilitate input of parameter values to appropriate fields. The tabular interface also contains fields for the power results which are then displayed in the spreadsheet following their calculation.

In some instances, it may be desirable to obtain power results for a range of parameter values so that the associated power values may be compared by the user for use in optimizing a genetic association study design. In such an instance, the spreadsheet may include a plurality of columns, each column containing a different set of values input for the study-defining parameters. Such a format also includes fields for the power results which are then displayed in the spreadsheet following their calculation for each set of parameter values.

The power results obtained for a range of parameters values (value sets) may also be advantageously displayed and viewed in a graphical form. For example, the power results may be plotted as a function of one or more parameters, such as allele frequency, heritability, dominance effect, number of cases, etc. Such a graphical presentation may facilitate the extraction of meaningful information from the study.

Use of the method and system of the present invention therefore enable the user to relatively easily apply sophisticated statistical analysis to genetic association study design in order to determine whether or not a study will provide a meaningful result before substantial resources are spent. The invention may also be applied to optimize a study design, e.g., maximize a study's power.

These and other features and advantages of the present invention are further described in the example, below.

### ALTERNATIVE EMBODIMENTS

In an alternative embodiment, the present invention may also be used to determine selected parameter values defining a genetic association study from a desired or required power for the study. In this embodiment, some (a subset) of the parameter values defining the study are entered in the spreadsheet together with a desired power value. The power algorithm is then used to determine the missing parameter value and complete the set defining the study. The set of parameter values defining the study is determined using an iterative refinement process until the desired power is obtained. In one embodiment, the “goal seek” function in Microsof Excel™ can be used to perform this operation.

### V. EXAMPLE

The following example provides details of case-control genetic association study analyses conducted in accordance with the present invention. The following example was conducted using the power algorithm described above implemented in a Microsoft Excel™ spreadsheet running on a Microsoft Windows™ based personal computer.

Dependence of Power on QTL Allele Frequency

FIG. 4A illustrates a column showing a baseline set a parameter values for a genetic association study involving a hypothetical gene. These baseline values are shown input (in the fourth column) into a computer-based spreadsheet programmed with a power algorithm in accordance with the method and system of the present invention in FIG. 4B. The power of a study having these parameters, 0.524, is calculated in accordance with the power algorithm and displayed in the bottom field of the column. The spreadsheet also includes several additional columns containing entries for a range of other values for the QTL frequency and Marker frequency parameters so that the effect of changing these parameters, which correspond to the allele frequency, can been seen. FIG. 4C is a plot of power vs. allele frequency illustrating the change of power depending upon QTL and marker frequencies for the hypothetical gene/marker pair. The data and graph enable the user to easily determine the study design with the most power for the given range of parameters.

Dependence of Power on QTL Effect Size

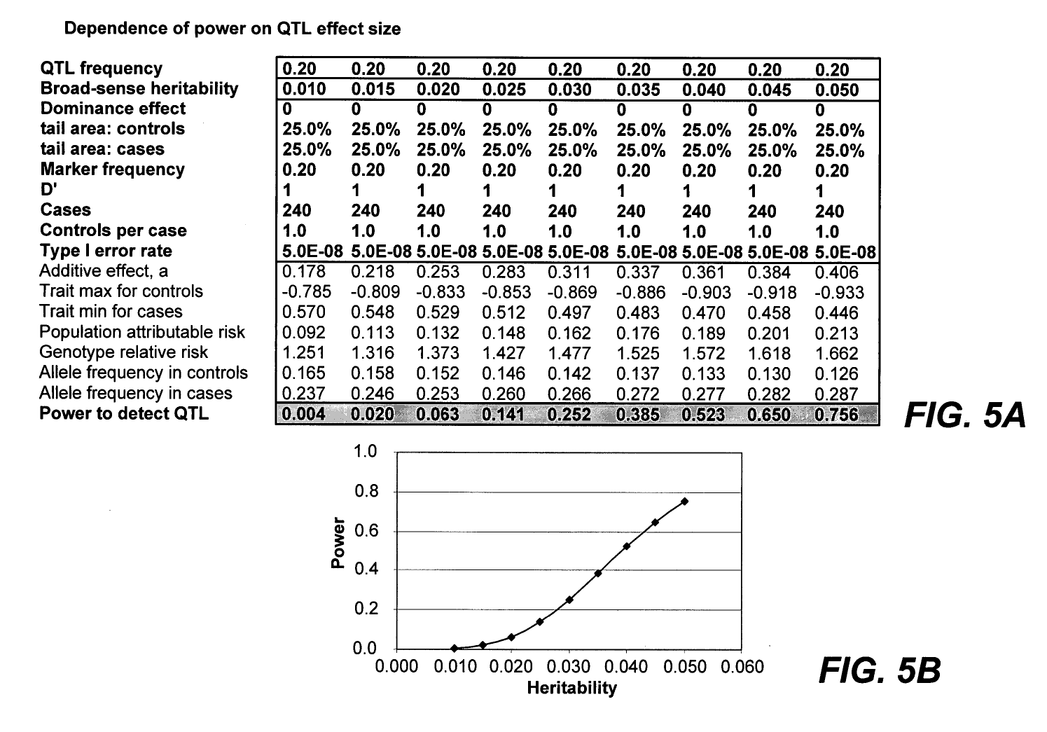

FIG. 5A illustrates sets of parameter values for a genetic association study involving a hypothetical gene input into the computer-based spreadsheet programmed with the power algorithm in accordance with the method and system of the present invention. The respective powers of studies having these parameters are calculated in accordance with the power algorithm and displayed in the bottom field of each column. The spreadsheet includes several columns containing entries for a range of values for the broad-sense heritability parameter so that the effect of changing this parameter can been seen. FIG. 5B is a plot of power vs. heritability illustrating the change of power depending upon broad-sense heritability for a hypothetical gene. The data and graph enable the user to easily determine the study design with the most power for the given range of parameters.

Dependence of Power on QTL Mode of Action

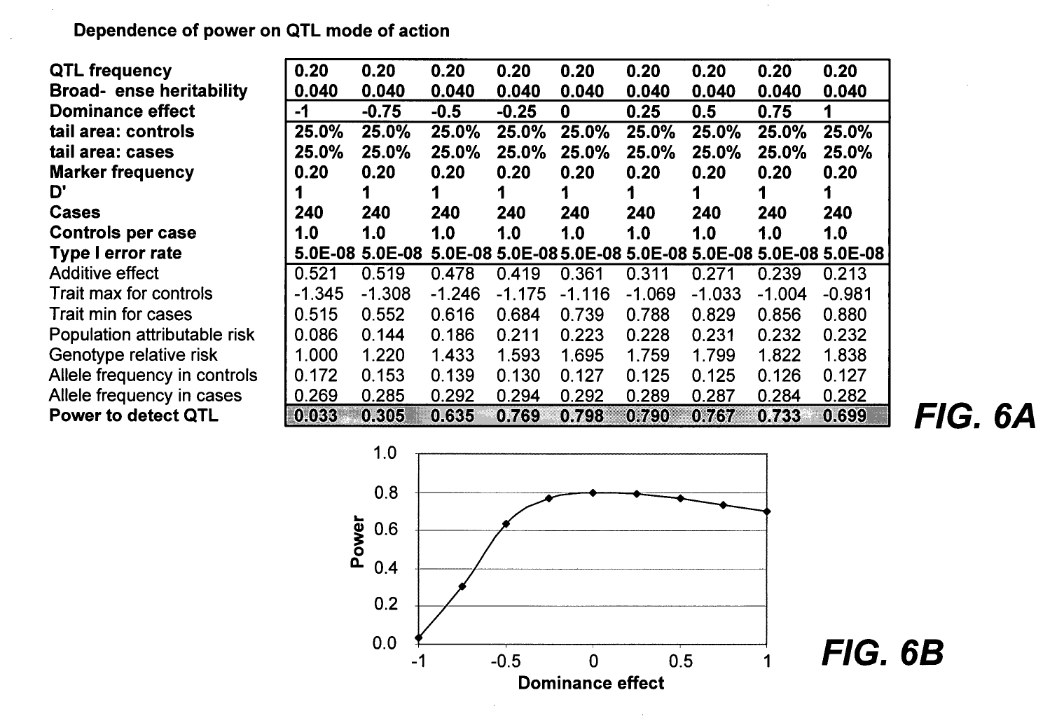

FIG. 6A illustrates sets of parameter values for a genetic association study involving a hypothetical gene input into the computer-based spreadsheet programmed with the power algorithm in accordance with the method and system of the present invention. The respective powers of studies having these parameters are calculated in accordance with the power algorithm and displayed in the bottom field of each column. The spreadsheet includes several columns containing entries for a range of values for the dominance effect parameter so that the effect of changing this parameter can been seen. FIG. 6B is a plot of power vs. dominance effect illustrating the change of power depending upon dominance effect for a hypothetical gene. The data and graph enable the user to easily determine the study design with the most power for the given range of parameters.

Dependence of Power on Number of Cases and Lewontin's D′

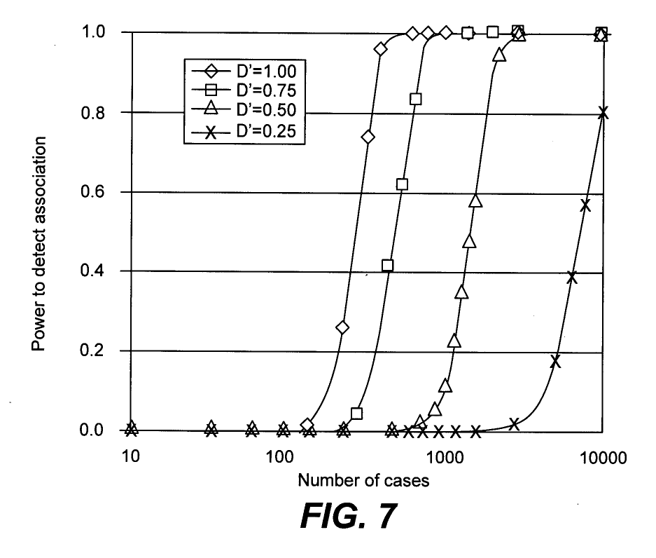

FIG. 7 is a plot of power vs. number of cases illustrating the change of power depending upon a measure of association between a hypothetical gene and its marker (Lewontin's D′). The data for the graph is obtained from a spreadsheet programmed with a power algorithm in accordance with the method and system of the present invention. The graph enables the user to see the relationship between the closeness of the association of the gene and marker, the number of cases and the power of the study.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Although the foregoing invention has been described in some detail for purposes of clarity of understanding, it will be apparent that certain changes and modifications may be practiced within the scope of the appended claims. Therefore, the present embodiments are to be considered as illustrative and not restrictive, and the invention is not to be limited to the details given herein, but may be modified within the scope and equivalents of the appended claims.

1. A computer implemented method for analyzing a case-control genetic association study, the method comprising:
(a) providing a spreadsheet program running on a computer;
(b) programming the spreadsheet software with a statistical power algorithm configured to analyze a case-control genetic association study;
(c) inputting to the spreadsheet program values for parameters defining the genetic association study; and
(d) determining, using the power algorithm, the study's power to detect a significant difference in distribution of observed allele frequency in cases and controls for the input parameter values.

2. The method of claim 1, wherein the determined power is displayed in the spreadsheet.

3. The method of claim 1, wherein a plurality of values are input for one or more parameters.

4. The method of claim 3, wherein the determined power is displayed in the spreadsheet.

5. The method of claim 4, further comprising displaying the power results obtained by the determination in graphical form.

6. The method of claim 5, wherein the graphical form is a bivariate plot.

7. The method of claim 1, wherein the parameters defining the genetic association study comprise trait value thresholds used to define case and controls; a number of cases; a ratio of controls to cases; and a desired type I error rate.

8. The method of claim 7, wherein the trait value thresholds used to define case and controls comprise a quantitative trait locus (QTL) frequency; broad sense heritability; dominance effect; case and control tail areas; marker frequencies; and a measure of association between marker and QTL sites.

9. A computer implemented method for analyzing a case-control genetic association study, the method comprising:
(a) providing a spreadsheet program running on a computer;
(b) programming the spreadsheet software with a statistical power algorithm configured to analyze a case-control genetic association study;
(c) inputting to the spreadsheet program a subset of values for parameters defining the genetic association study;
(d) inputting to the spreadsheet program a desired power of the study to detect a significant difference in distribution of observed allele frequency in cases and controls; and
(e) determining, using the power algorithm, a complete set of values for parameters defining the genetic association study.

10. The method of claim 9, wherein the parameters defining the genetic association study comprise trait value thresholds used to define case and controls; a number of cases; a ratio of controls to cases; and a desired type I error rate.

11. The method of claim 10, wherein the trait value thresholds used to define case and controls comprise a quantitative trait locus (QTL) frequency; broad sense heritability; dominance effect; case and control tail areas; marker frequencies; and a measure of association between marker and QTL sites.

12. The method of claim 9, wherein the input subset of values excludes a number of cases.

13. The method of claim 12, wherein the complete set of values includes a number of cases.

14. The method of claim 9, wherein determining the complete set of values is an iterative refinement process.

15. A computer program product comprising a computer-usable medium having computer-readable program code embodied thereon relating to analyzing a case-control genetic association study, the computer program product comprising computer-readable program code for effecting the following steps within a computing system:
(a) configuring a spreadsheet program running on a computer with a statistical power algorithm for analysis of a case-control genetic association study;
(b) receiving to the spreadsheet program values for parameters defining the genetic association study; and
(c) determining, using the power algorithm, the study's power to detect a significant difference in distribution of observed allele frequency in cases and controls for the input parameter values.

16. The computer program product of claim 13, wherein the computer-usable medium comprises at least one of a magnetic medium, an optical medium, a hardware device specially configured to store and perform program instructions, and a carrier wave.

17. The computer program product of claim 13, wherein the parameters defining the genetic association study comprise trait value thresholds used to define case and controls; a number of cases; a ratio of controls to cases; and a desired type I error rate.

18. The computer program product of claim 17, wherein the trait value thresholds used to define case and controls comprise a quantitative trait locus (QTL) frequency; broad sense heritability; dominance effect; case and control tail areas; marker frequencies; and a measure of association between marker and QTL sites.

19. A computer program product comprising a computer-usable medium having computer-readable program code embodied thereon relating to analyzing a case-control genetic association study, the computer program product comprising computer-readable program code for effecting the following steps within a computing system:
(a) configuring a spreadsheet program running on a computer with a statistical power algorithm for analysis of a genetic association study;
(b) receiving to the spreadsheet program a subset of values for parameters defining the case-control genetic association study;
(c) receiving to the spreadsheet program a desired power of the study to detect a significant difference in distribution of observed allele frequency in cases and controls; and
(d) determining, using the power algorithm, a complete set of values for parameters defining the genetic association study.

20. The computer program product of claim 19, wherein the computer-usable medium comprises at least one of a magnetic medium, an optical medium, a hardware device specially configured to store and perform program instructions, and a carrier wave.

21. The computer program product of claim 20, wherein the parameters defining the genetic association study comprise trait value thresholds used to define case and controls; a number of cases; a ratio of controls to cases; and a desired type I error rate.

22. The computer program product of claim 21, wherein the trait value thresholds used to define case and controls comprise a quantitative trait locus (QTL) frequency; broad sense heritability; dominance effect; case and control tail areas; marker frequencies; and a measure of association between marker and QTL sites.

23. A computer system for analyzing a case-control genetic association study, the computer system, comprising:
(a) a computer;
(b) a spreadsheet program running on the computer with a statistical power algorithm for analysis of a case-control genetic association study, the spreadsheet program configured to,
(i) receive program values for parameters defining the genetic association study, and
(ii) determine, using the power algorithm, the study's power to detect a significant difference in distribution of observed allele frequency in cases and controls for the input parameter values.

24. The system of claim 23, wherein the parameters defining the genetic association study comprise trait value thresholds used to define case and controls; a number of cases; a ratio of controls to cases; and a desired type I error rate.

25. The system of claim 24, wherein the trait value thresholds used to define case and controls comprise a quantitative trait locus (QTL) frequency; broad sense heritability; dominance effect; case and control tail areas; marker frequencies; and a measure of association between marker and QTL sites.

26. A computer system for analyzing a case-control genetic association study, the computer system, comprising:
(a) a computer;
(b) a spreadsheet program running on the computer with a statistical power algorithm for analysis of a case-control genetic association study, the spreadsheet program configured to,
(i) receive a subset of values for parameters defining the genetic association study;
(ii) receive a desired power of the study to detect a significant difference in distribution of observed allele frequency in cases and controls; and
(iii) iteratively determine, using the power algorithm, a complete set of values for parameters defining the genetic association study having the desired power.

27. The system of claim 26, wherein the parameters defining the genetic association study comprise trait value thresholds used to define case and controls; a number of cases; a ratio of controls to cases; and a desired type I error rate.

28. The system of claim 27, wherein the trait value thresholds used to define case and controls comprise a quantitative trait locus (QTL) frequency; broad sense heritability; dominance effect; case and control tail areas; marker frequencies; and a measure of association between marker and QTL sites.
